(19) United States
(12) Patent Application Publication
(10) Pub. No.: US 2005/0021067 A1
(43) Pub. Date: Jan. 27, 2005

(54) Accupuncture needle insertion tube

(75) Inventor: Kuen Sik Kim, Chungnam (KR)

Correspondence Address: SUGHRUE MION, PLLC, 2100 PENNSYLVANIA AVENUE, N.W., SUITE 800, WASHINGTON, DC, 20037, US

(73) Assignees: Lhasa OMS, Inc.; Dong Bang Acupuncture, Inc.

(21) Appl. No.: 10/626,544

(22) Filed: Jul. 25, 2003

(51) Int. Cl. A61B 17/34; A61B 17/16

(52) U.S. Cl. 606/189; 606/96

(57) ABSTRACT

An acupuncture needle insertion guide tube including a guide tube body having a proximal end portion, a distal end portion, an inner diameter, and an outer surface. At the proximal end portion, there is disposed a needle holding structure monolithically formed with the guide tube body. The needle holding structure includes a needle holding clamp, and a needle passing opening, wherein the needle holding clamp includes a needle holding opening that is smaller than the needle passing opening. The needle holding opening is sized to hold an acupuncture needle handle, whereas the needle passing opening is sized to freely pass the acupuncture needle handle.

### FIELD OF THE INVENTION

The present invention relates to the field of needle therapy, specifically to an acupuncture needle assembly with an improved needle guide tube.

### BACKGROUND OF THE INVENTION

The field of acupuncture has recently grown in popularity with the embrace of alternative medicines. This ancient practice uses very thin stainless steel needles. During the insertion into the skin a guide tube is used for holding the acupuncture needle. The function of the guide tube is twofold, to allow for placement as well as protection of the acupuncture needle. This guide tube has been modified many times over the years, with the goal of increasing the accurate placement of the acupuncture needle on the skin. Accurate placement of the needle on the skin allows for proper insertion through the epidermis and to the patient's acupuncture point. Currently, the acupuncture needle is held in a guide tube with a separate holding tab. The holding tab is wedged between the acupuncture needle and the guide tube, thus preventing the acupuncture needle from slipping out of the tube. The whole assembly is then packaged into a sterile disposable package.

Once the holding tab is removed by the practitioner the acupuncture needle is free to move within the insertion guide tube. To prevent the acupuncture needle from prematurely slipping from the guide tube, the practitioner will hold or press the handle end of the acupuncture needle to one side of the guide tube. The acupuncture and guide tube assembly are then placed with moderate pressure on the skin of the patient. The pressure exerted by the guide tube is designed to pull the skin tight, and allow for fast, pain-free insertion. The handle of the acupuncture needle is then tapped with a finger to insert the needle into the body. Unfortunately, the relatively large inner diameter of the guide tube with respect to the diameter of the acupuncture needle allows for a great deal of needle movement within the guide tube. This movement can compromise the precision of insertion.

Difficulty with the conventional assembly is not simply directed at accurate placement. In addition, once the holding tab is removed, and the needle is free to move, the practitioner may have difficulty maintaining the needle in the guide tube. This may result in the acupuncture needle falling out of the guide tube onto the ground. In such a case, the needle would no longer be sterile, and would have to be discarded.

### SUMMARY OF THE INVENTION

One aspect consistent with the present invention is to provide a new insertion guide tube for holding and inserting an acupuncture needle. This aspect eliminates the need for a separate wedge. By using a holding structure monolithically formed with the guide tube, accurate and convenient insertion can be achieved. The holding structure maintains the needle in position when not in use, cutting down on accidental release from the needle insertion tube. The holding structure also allows for easy mass production, which reduces manufacturing costs.

### BRIEF DESCRIPTION OF THE DRAWINGS

The accompanying drawings, which are included to provide a further understanding of illustrative, non-limiting embodiments of the present invention and are incorporated in and constitute a part of this specification, illustrate embodiments of the invention and together with the description serve to explain the principles of the drawings.

FIG. 1 is an exploded view of both an acupuncture needle and a guide tube according to the concepts of the present invention;

FIG. 2 is a view of an acupuncture needle held in place within a guide tube according to the concepts of the present invention;

FIGS. 3A and 3B are cross sectional views of a proximal end of a guide tube holding structure according to the concepts of the present invention;

FIGS. 4A, 4B, and 4C, are cross sectional views of a proximal end of a guide tube showing several embodiments of the holding structure according to the concepts of the present invention;

FIG. 5 is a view of an acupuncture needle and guide tube disposed within a resinoid package according to the concepts of the present invention; and

FIGS. 6A and 6B are views of an acupuncture needle and guide tube at an acupuncture point on a patient's skin.

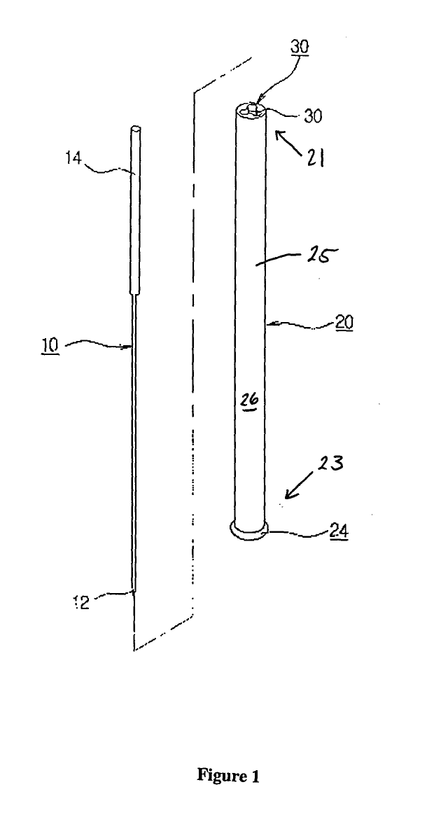

### DETAILED DESCRIPTION OF ILLUSTRATIVE, NON-LIMITING EMBODIMENTS

Reference will now be made in detail to the illustrative, non-limiting embodiments of the present invention, examples of which are illustrated in the accompanying drawings. The terms are meant to have the definition provided in the specification, and are otherwise not limited by the specification. Further advantages of these and the stated objects reside in the details of construction and operation as more fully hereinafter described and claimed, reference being made to the accompanying drawings forming a part hereof, wherein like numerals refer to like parts throughout.

An acupuncture device according to concepts consistent with the present invention comprises an acupuncture needle 10 and a guide tube 20. Description of the needle 10 and guide tube 20 will be made with reference to the figures.

The acupuncture needle 10 is a stainless steel needle that comprises a needle body which is made of long thin stainless steel. At the distal end of the needle, there is a pointed tip 12; a polished sharp point that is to be inserted at an acupuncture point. At the proximal end of the acupuncture needle, there is a handle 14 that can be a ridged, helical coil, or a smooth tube. For example, but not by way of limitation, a ridged, helical coil or a smooth type handle can be used with the guide tube 20 that is structured according to concepts of the present invention. The handle 14 may be made of any suitable material as, by way of non-limiting example, stainless steal, plastic and the like.

An insertion guide tube 20 has a body 25 including a proximal end portion 21 and a distal end portion 23, and an inner diameter 22. During use of the needle 10 and insertion guide tube 20, the proximal end portion 21 is located closest to the practitioner and the distal end portion 25 is located closest to the patient's skin S. The insertion guide tube 20 can be made of any suitable material as, by way of non-limiting example, plastic, metal or the like. If made of plastic, polyethylene or polypropylene may be used, for example.

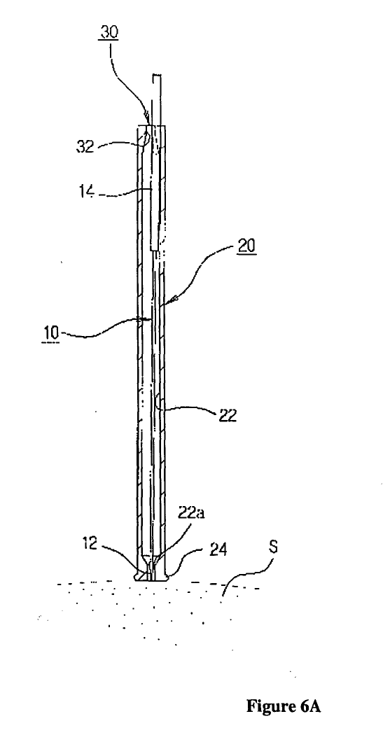

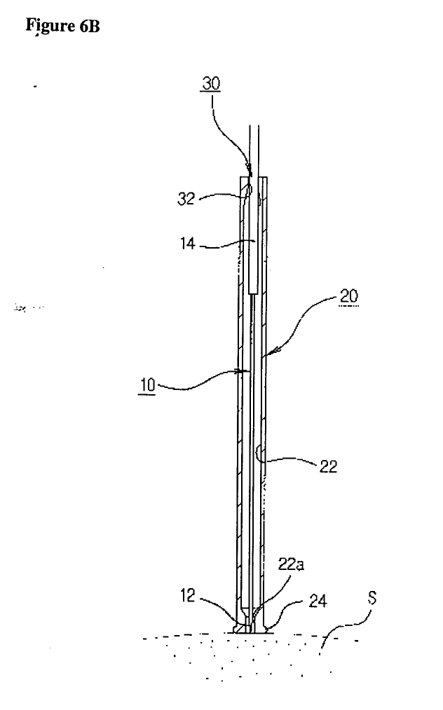

The proximal end portion 21 includes a holding structure 30 monolithically formed with the guide tube body 25. The holding structure 30 monolithically is molded directly into the guide tube body 25, and includes a needle holding clamp—having a needle holding opening 32—as well as a needle passing opening 34. The needle holding opening 32 is sized snugly to fit the handle portion 14 of an acupuncture needle and may be disposed so as to extend into the interior of the guide tube body 25. According to another structure consistent with the present invention, due to the resilience of the material from which the guide tube 20 is made and to the relative diameter of the opening 32 with respect to the diameter of the needle handle 14, the needle holding opening 32 acts as a clamp to securely hold the needle handle 14. If a needle handle 14 having a ridged coil structure is used, there can be achieved a more slip-resistant fit with the needle holding opening 32. FIG. 6A shows the needle guide tube 20 positioned at an acupuncture point on a patient's skin S, with needle handle 14 in needle holding opening 32. The needle passing opening 34 is sized so as to freely pass a needle handle 14 therethrough. For example, when insertion is appropriate, the handle 14 of the acupuncture needle 10 is moved from the needle holding opening 32 and is placed into the needle passing opening 34. Detaching the needle handle 14 from the holding opening 32 and moving it to the needle passing opening 34 requires only modest force. This needle passing opening 34 then allows for the release of the acupuncture needle 10 when the practitioner has determined the proper position on the skin S. FIG. 6B shows the needle guide tube 20 positioned at an acupuncture point on a patient's skin S, with the needle handle 14 moved from needle holding opening 32 to needle passing opening 34.

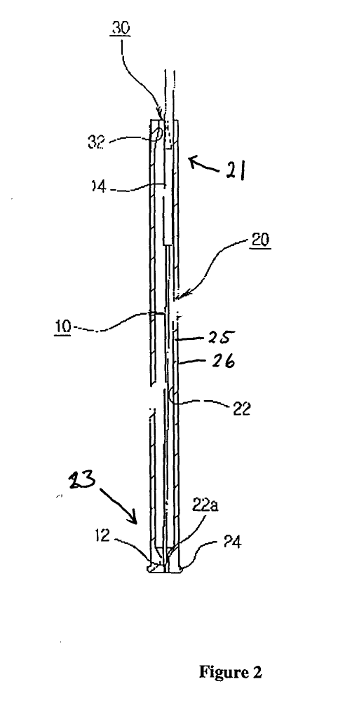

FIG. 2 shows the needle 10 securely positioned in the guide tube 20. This secure positioning of the needle 10 is obtained at the proximal end 21 of the guide tube 20 by the holding structure 30 and, more specifically, by the needle 10 snugly fitting in the needle holding opening 32. The needle holding opening 32 thus prevents early release of the acupuncture needle, allowing greater control by the practitioner. The thusly configured guide tube 20 also allows for increased precision in insertion of the needle 10 into the skin S of a patient.

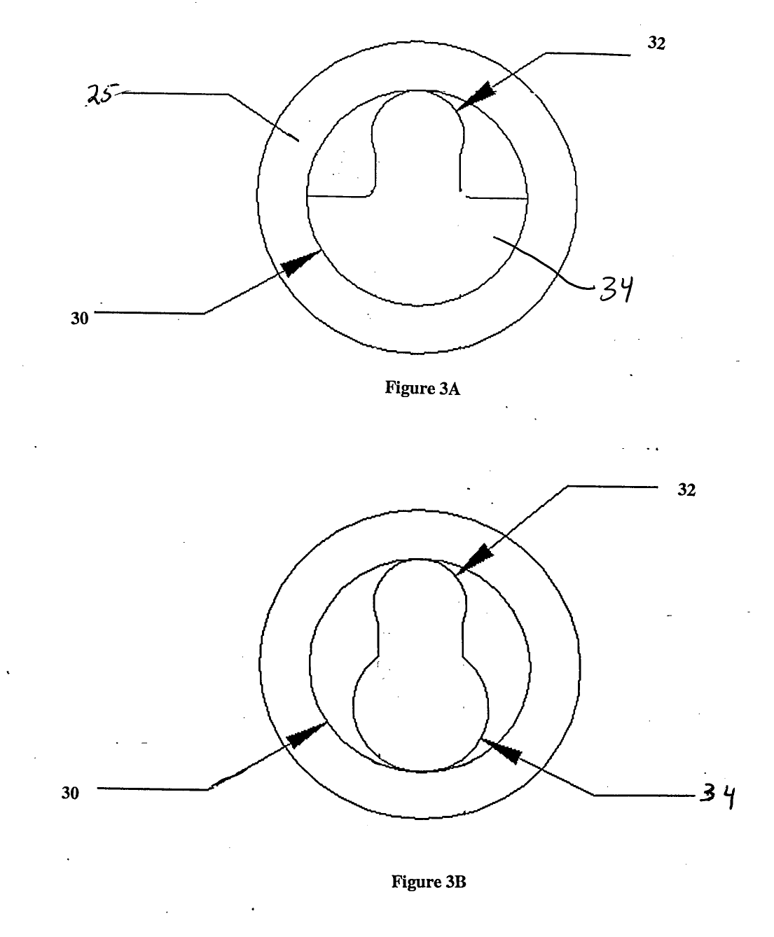

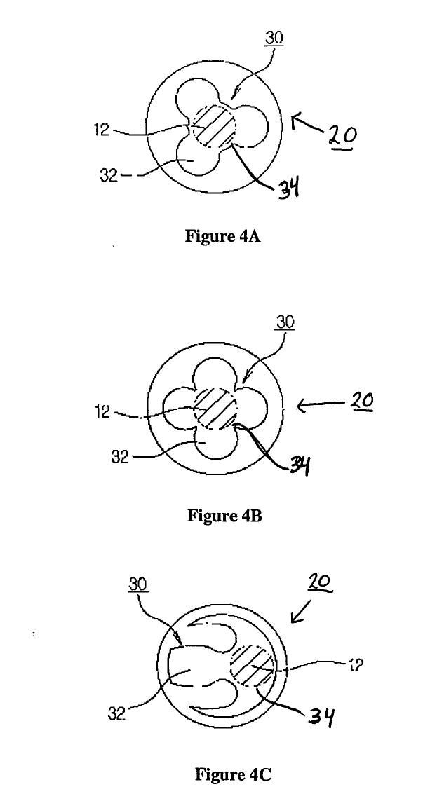

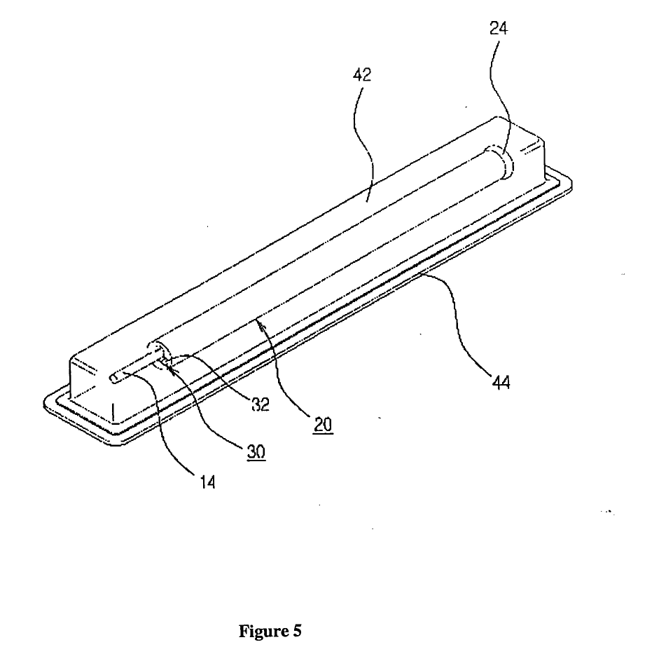

FIGS. 3A and 3B show, in a profile view, non-limiting exemplary embodiments of the needle holding structure 30 at the proximal end portion 21. FIGS. 4A-C, show several other different exemplary embodiments of the needle holding structure 30 at the proximal end portion 21 of the needle guide tube 20. Each embodiment comprises at least one needle holding opening 32 and at least one needle passing opening 34 that allows for precise insertion of the needle 10 to an acupuncture point at the patient's skin S. Although the drawings show only one, three, and four needle holding openings 32 in any one embodiment, alternately there may be any suitable number of such needle holding openings 32 in any one needle insertion guide tube 20. One consideration when designing multiple needle holding openings 32 is that they preferably are located at the same distance from the center of the needle insertion guide tube and overlap with the needle passing opening 34. Also, although each embodiment is shown with only one needle passing opening 34, alternately there may be any suitable number of such needle passing openings 34 in any one needle insertion guide tube 20.

As shown in FIG. 4A for example, but not by way of limitation, one embodiment consistent with concepts of the present invention includes a needle holding structure 30 having three needle holding openings 32 that are connected to one needle passing opening 34. The needle holding openings 32 are located at an equal distance from the center of the insertion guide tube 20.

As shown in FIG. 4B, an alternative embodiment consistent with concepts of the present invention includes a needle holding structure 30 having four needle holding openings 32 that are connected to one needle passing opening 34. The arrangement in FIG. 4B facilitates insertion of the needle 10 into one of the holding openings 32, as the number of possible holding openings 32 is increased.

Although the needle holding openings 32 shown in FIGS. 4A and 4B are circular as an example but not by way of limitation, they need not necessarily have such a shape, as long as they snugly hold the needle handle 14. FIG. 4C specifically shows a needle holding opening 32 that is not circular.

According to another embodiment consistent with concepts of the present invention, the distal end portion 23 of the guide tube 20 may include a tapered portion 22a monolithically formed on the inside thereof, and a rib 24 on the outside surface 26 of the guide tube.

The inside of the distal end portion 23 has an opening that is sized such that the acupuncture needle 10, including the handle 14, can pass therethrough. Additionally, at the distal end portion 23, the guide tube 22 may include a tapered portion 22a, monolithically formed therein, which narrows as it extends distally. The tapered portion 22a, monolithically formed in the guide tube 20, is sized such that the acupuncture needle tip 12 and handle 14 may pass therethrough, but is not sized so large as to allow for excess play between the needle 10 and the guide tube 20. The tapered portion 22a may be shaped like a funnel, which allows for the passage of the entire needle; the opening in the tapered portion 22a is large enough to allow for the passage of the needle handle 14, but more accurately guides the needle tip 12 to a correct acupuncture point.

The rib 24, on the outside surface 26 of the distal end portion 23, for example, but not by way of limitation, allows for a machine to differentiate between the proximal 21 and distal 23 ends of the guide tube 20. This differentiation allows for automated assembly of the acupuncture device. The machinery is able to insert the needle 10 at the proximal end 21, and maintain the position of the needle 10 securely in the holding structure 30.

Once the needle 10 is assembled to the guide tube 20, the assembly is wrapped in a packaging. This packaging may be made of a blister pack, for example. The blister pack includes, by way of non-limiting example, a resinoid shroud 42 attached to a sterile paper 44, with the needle-and-guide-tube assembly therebetween. Then, the entire package is sterilized.

As can be seen from the above, embodiments consistent with concepts of the present invention reliably secure an acupuncture needle in an insertion guide tube. The guide tube also allows for precise placement of the acupuncture needle at the proper acupuncture point, as the needle passes through the guide tube and into a patient's skin. Embodiments according to concepts of the present invention thus provide an acupuncture needle insertion guide tube that is of a simple, single-unit, construction that allows for easy needle manipulation, and ease of manufacture. Further, the needle and guide tube are much easier to assemble, and also to use when inserting the needle into an acupuncture point on a body.

Although the invention has been described with reference to the foregoing exemplary, non-limiting, embodiments, it is understood that the invention is not limited by these embodiments and that any changes and modifications are possible, provided they do not depart from the scope of the attached patent claims. It will be appreciated that the present invention is not limited to the foregoing exemplary embodiments, and that various changes and modifications can be effected therein by one of ordinary skill in the art without departing from the scope or spirit of the invention defined by the claims.

1. An needle insertion guide tube comprising:
a guide tube body comprising a proximal end, a distal end, an inner diameter, and an outer surface;
said proximal end comprising a needle holding structure monolithically formed with said guide tube body, wherein said needle holding structure comprises a needle holding clamp, and a needle passing opening, wherein said needle holding clamp comprises a needle holding opening that is smaller than said needle passing opening, and further wherein said needle holding opening is sized to hold an acupuncture needle handle, and said needle passing opening is sized to freely pass the acupuncture needle handle.

2. The needle insertion guide tube according to claim 1, further comprising a rib formed on the outer surface adjacent said distal end.

3. The needle insertion guide tube according to claim 1, wherein said distal end comprises a distal needle passing structure monolithically formed with said guide tube body, wherein said distal needle passing structure comprises a tapered structure such that the inner diameter is narrowed at the distal end, wherein said distal needle passing structure is sized and configured not smaller than the handle of an acupuncture needle.

4. The needle insertion guide tube according to claim 1, wherein said needle holding structure further comprises a second needle holding clamp and a third needle holding clamp.

5. The needle insertion guide tube according to claim 4, wherein said needle holding structure further comprises a fourth needle holding clamp.

6. A needle insertion guide tube comprising:
a guide tube body comprising a proximal end, a distal end, an inner diameter, and an outer diameter;
said distal end comprising a distal needle passing structure monolithically formed with said guide tube body, wherein said distal needle passing structure comprises a tapered structure such that the inner diameter is narrowed at the distal end, wherein said distal needle passing structure is sized and configured not smaller than the handle of an acupuncture needle.

7. The needle insertion guide tube according to claim 6, wherein said proximal end comprises a needle holding structure monolithically formed with said guide tube body, wherein said needle holding structure comprises a needle holding clamp, and a needle passing opening, wherein said needle holding clamp comprises a needle holding opening that is smaller than said needle passing opening, and further wherein said needle holding opening is sized to hold an acupuncture needle handle, and said needle passing opening is sized to freely pass the acupuncture needle handle.

8. An acupuncture needle assembly comprising:
a guide tube body comprising a proximal end, a distal end, an inner diameter, and an outer surface;
said proximal end comprising a needle holding structure monolithically formed with said guide tube body, wherein said needle holding structure comprises a needle holding clamp, and a needle passing opening, wherein said needle holding clamp comprises a needle holding opening that is smaller than said needle passing opening, and further wherein said needle holding opening is sized to hold an acupuncture needle handle, and said needle passing opening is sized to freely pass the acupuncture needle handle;
an acupuncture needle comprising a proximal handle, a needle body, and a polished point, wherein said acupuncture needle is disposed in said needle holding clamp.

9. An acupuncture needle assembly according to claim 8, wherein said acupuncture needle proximal handle comprises a helical coil, wherein said helical coil handle is ridged with concentric circles.

10. An acupuncture needle assembly according to claim 8, wherein said distal end comprises a distal needle passing structure monolithically formed with said guide tube body, wherein said distal needle passing structure comprises a tapered structure such that the inner diameter is narrowed at the distal end, wherein said distal needle passing structure is sized and configured not smaller than the handle of an acupuncture needle.

11. An acupuncture needle assembly according to claim 8, wherein the acupuncture needle and needle guide tube are packaged in a wrapping,
wherein said wrapping is made from a resinoid, that is attached to a sterile purpose paper.

12. An acupuncture needle assembly according to claim 9, wherein the acupuncture needle and needle guide tube are packaged in a wrapping,
wherein said wrapping is made from a resinoid, that is attached to a sterile purpose paper.
